(19) United States
(12) Patent Application Publication
(10) Pub. No.: US 2008/0204021 A1
(43) Pub. Date: Aug. 28, 2008

(54) Flexible and Wearable Radio Frequency Coil Garments for Magnetic Resonance Imaging

(75) Inventors: Christoph G. Leussler, Hamburg (DE); Ingmar Graesslin, Boenningstedt (DE); Hans Buurman, 'S-Hertogenbosch (NL); John T. Carlon, Madison, OH (US)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N.V., Eindhoven (NL)

(21) Appl. No.: 11/570,507

(22) PCT Filed: Jun. 2, 2005
(86) PCT No.: PCT/IB05/51806

§ 371 Date: Dec. 17, 2007

Related U.S. Application Data
(60) Provisional application No. 60/580,619, filed on Jun. 17, 2004.

(51) Int. Cl. G01R 33/3415; A61B 5/055

(52) U.S. Cl. 324/318; 600/422

(57) ABSTRACT

A radio frequency apparatus for at least one of (i) receiving and (ii) exciting a magnetic resonance signal includes an item of clothing (102, 202). The item of clothing includes one or more layers (110, 112, 120, 122, 300, 402) that are stretchable to comport with differently sized and shaped imaging subjects. A plurality of radio frequency coils (104, 114, 204, 206, 208, 302, 404) are attached to one or more layers of the item of clothing. The coils are relatively movable with respect to one another responsive to stretching of the stretchable item of clothing. The one or more layers of the item of clothing include an anti-microbial agent (92, 92′, 94′) disposed on or incorporated into at least one layer.

The following relates to the magnetic resonance arts. It finds particular application in safe, patient-friendly magnetic resonance imaging, and will be described with particular reference thereto. However, it also finds application in magnetic resonance spectroscopy and related magnetic resonance techniques.

The use of arrays of surface coils is becoming more prevalent in magnetic resonance imaging as multiple-coil imaging techniques such as phased-array imaging, SENSE imaging, and the like gain popularity. The use of surface coils introduces certain difficulties, however. In multi-coil techniques, a large number of coils may be used. Positioning many surface coils in close proximity to the patient can be difficult and uncomfortable to the patient. Moreover, the radio frequency surface coils are generally not familiar items for the patient, and being surrounded and/or contacted by a large number of surface coils can be intimidating and stressful for the patient. The coils are typically made of a plastic or other material that is uncomfortable when placed in contact with the patient, and which does not “breathe” to allow air to reach the patient's skin. In some coil arrangements, the coils rest on the patient, so that the weight of the coils is supported by the patient. This can be uncomfortable for the patient since the coils may weigh 15 kilograms or more.

Surface coils in contact with or in close proximity to the imaging subject may also be susceptible to becoming contaminated by blood, urine, vomit, or other body fluids excreted from a human imaging subject. Although the coils are generally cleaned before use, soiling from body fluids may not be completely removed. Moreover, the surface coils provide a potential vector for transmitting infectious organisms between patients or between a patient and the radiologist, technician, or other scanner operator. Disinfecting the coils, for example by using a Clorox solution, may not kill all pathogens. The problem of spread of infectious pathogens is not limited to the surface coils. Indeed, any surface with which the patient or radiologist comes into contact can become a vector for transmission of pathogens.

The present invention contemplates improved apparatuses and methods that overcomes the aforementioned limitations and others.

According to one aspect, a radio frequency apparatus is disclosed for at least one of (i) receiving and (ii) exciting a magnetic resonance signal. An item of clothing includes one or more layers that are stretchable to comport with differently sized and shaped imaging subjects. A plurality of radio frequency coils are attached to one or more layers of the item of clothing. The coils are relatively movable with respect to one another responsive to stretching of the stretchable layers.

According to another aspect, a radio frequency apparatus is disclosed for at least one of (i) receiving and (ii) exciting a magnetic resonance signal. At least one radio frequency antenna is provided. A structure is disposed on or around the at least one radio frequency antenna. The structure includes an anti-microbial agent disposed on or incorporated into the structure.

According to yet another aspect, a magnetic resonance imaging scanner is disclosed for imaging an imaging subject. A main magnet housed in a gantry generates a substantially spatially and temporally constant magnetic field in an examination region. Magnetic field gradient coils housed in the gantry generate selected magnetic field gradients in the examination region. A subject support supports the subject in the examination region. At least one radio frequency coil is arranged proximate to the imaging subject in the examination region. An operator control is contacted by an associated scanner operator. An anti-microbial agent is disposed on or incorporated into at least one of (i) the at least one radio frequency coil, (ii) the gantry, (iii) the subject support, and (iv) the operator control.

One advantage resides in improved patient safety during magnetic resonance imaging due to a reduced likelihood of infection and reduced patient stress.

Another advantage resides in providing a radio frequency coil array that is comfortable for various differently sized and shaped patients and which covers the anatomical region of interest.

Yet another advantage resides in providing a radio frequency coil array that is easily and accurately positioned.

Numerous additional advantages and benefits will become apparent to those of ordinary skill in the art upon reading the following detailed description of the preferred embodiments.

The invention may take form in various components and arrangements of components, and in various process operations and arrangements of process operations. The drawings are only for the purpose of illustrating preferred embodiments and are not to be construed as limiting the invention.

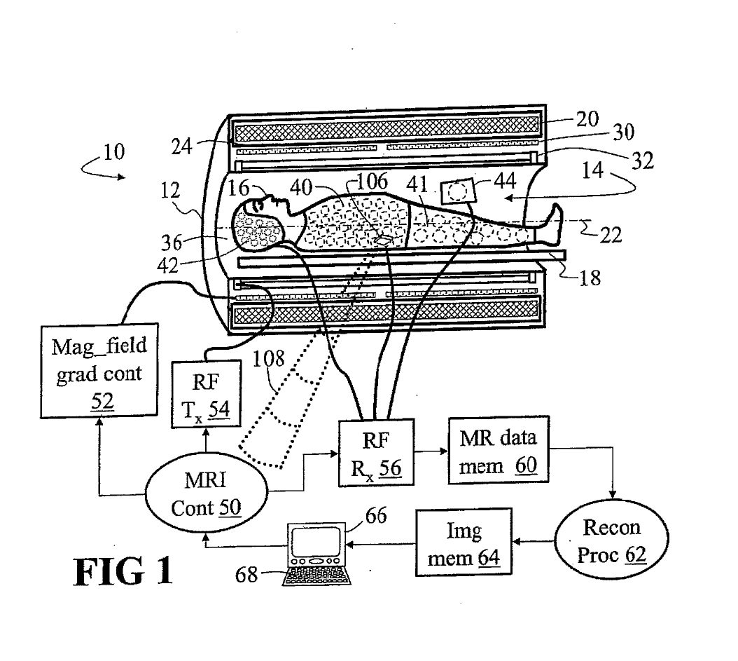

FIG. 1 diagrammatically shows a magnetic resonance imaging system including a plurality of radio frequency surface coils embodied as clothing apparel.

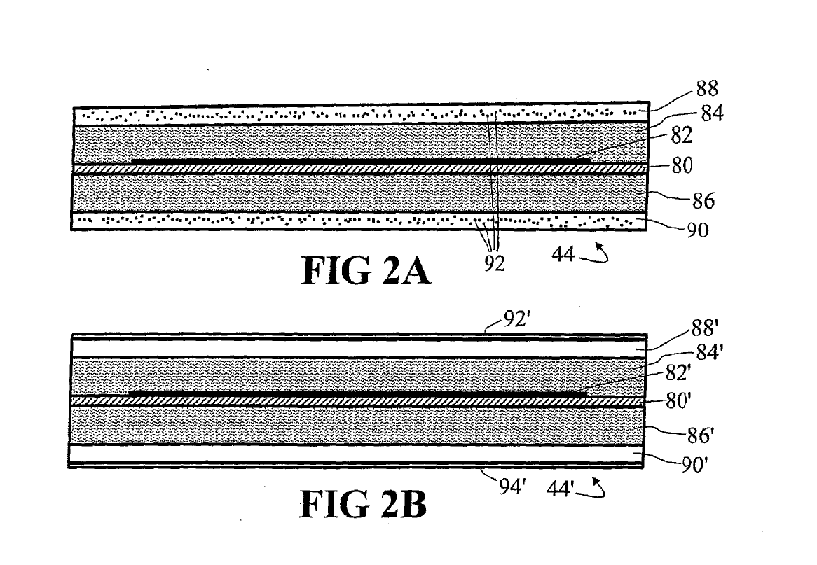

FIGS. 2A and 2B show diagrammatic cross-sectional views of two embodiments of the anti-microbial radio frequency surface coils of FIG. 1.

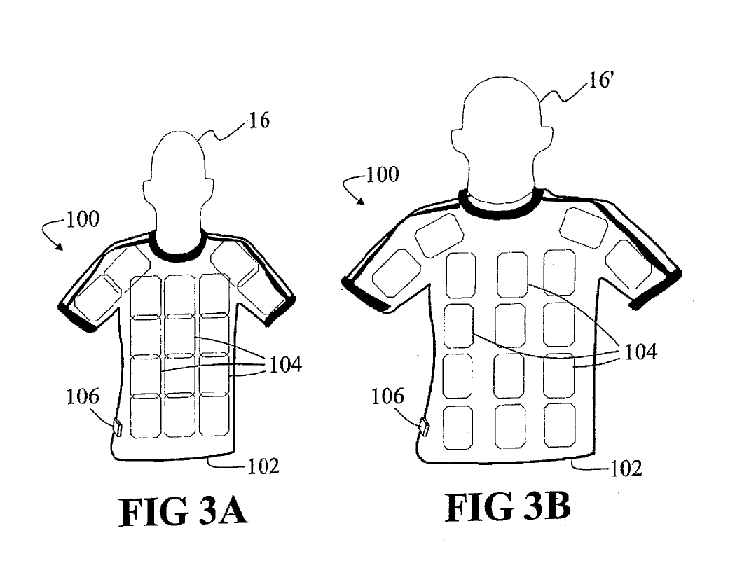

FIGS. 3A and 3B diagrammatically show a wearable surface coil array designed as a wearable shirt. FIG. 3A shows the shirt worn by a small, thin imaging subject, while FIG. 3B shows the shirt worn by a larger, more robust imaging subject.

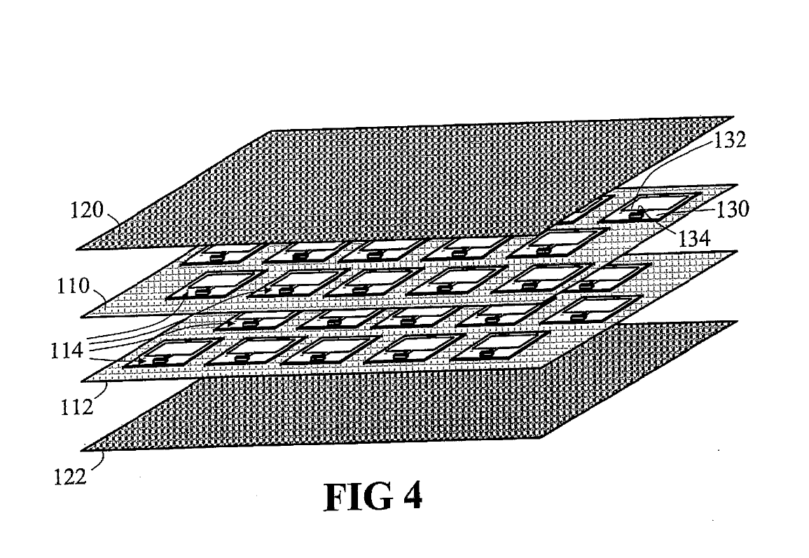

FIG. 4 diagrammatically shows an exploded perspective view of a portion of a wearable surface coil array.

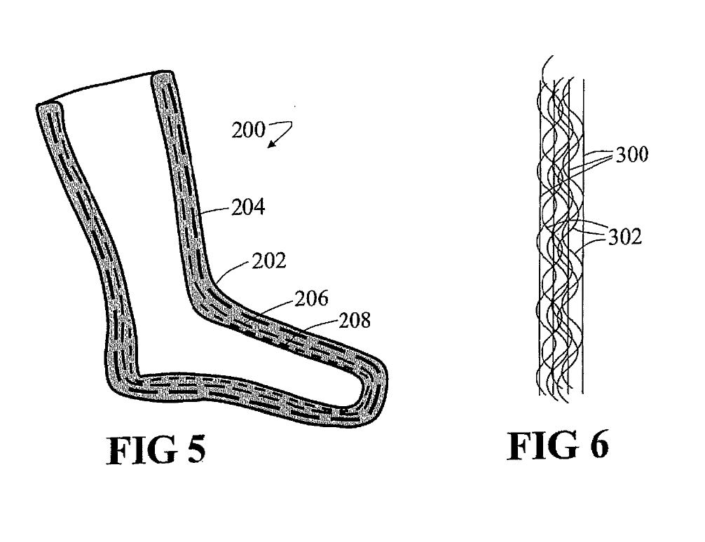

FIG. 5 diagrammatically shows a cross-sectional view of three sets of wearable surface coil arrays designed as a wearable sock.

FIG. 6 diagrammatically shows an alternative approach for integrating radio frequency coils with wearable fabric, in which the coils are embodied by conductive fibers embedded in the clothing.

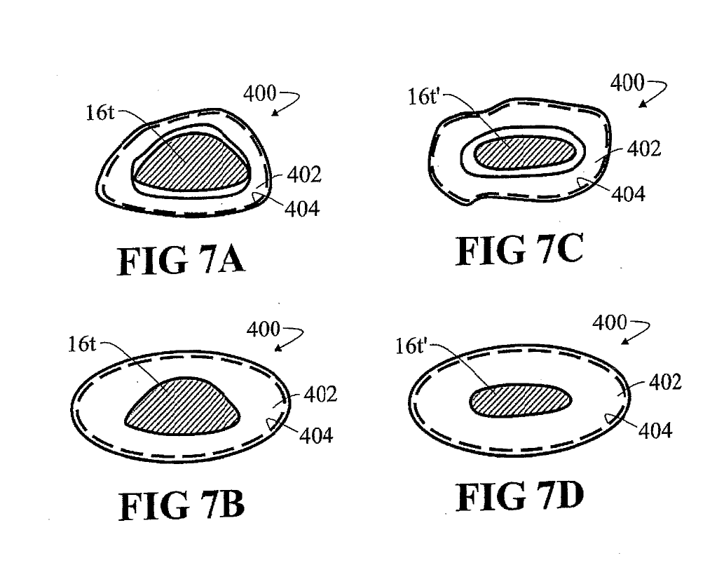

FIGS. 7A and 7B diagrammatically show a transverse sectional view of an inflatable vest coil array disposed on a large, rotund imaging subject.

FIGS. 7C and 7D diagrammatically show a transverse sectional view of an inflatable vest coil array disposed on a small, thin imaging subject.

With reference to FIG. 1, a magnetic resonance imaging scanner 10 includes a gantry or housing 12 defining a generally cylindrical scanner bore 14 inside of which an associated imaging subject 16 is disposed on a pallet or subject support 18. Main magnetic field coils 20 are disposed inside the housing 12, and produce a main B₀ magnetic field directed generally parallel with a central axis 22 of the scanner bore 14. The main magnetic field coils 20 are typically superconducting coils disposed inside cryoshrouding 24, although resistive main magnets can also be used. The housing 12 also houses or supports magnetic field gradient coils 30 for selectively producing magnetic field gradients in the bore 14. The housing 12 further houses or supports a radio frequency body coil 32 for selectively exciting magnetic resonances. The housing 12 typically includes a cosmetic inner liner 36 defining the scanner bore 14.

One or more radio frequency surface coils are disposed inside the bore 14 close to or in contact with the imaging subject 16. In some embodiments, a plurality of radio frequency surface coils are attached to or embedded in an item of clothing apparel. In FIG. 1, for example, a stretchable radio frequency coil apparel 40 has coils embedded in a shirt. A stretchable radio frequency coil apparel 41 has coils embedded in trousers. A stretchable radio frequency coil apparel 42 has coils embedded in a cap. The plurality of surface coils 40, 41, 42 can be used as a phased array of receivers for parallel imaging, as a sensitivity encoding (SENSE) coil array for acquiring SENSE imaging data, or the like. In another approach, the coils are used to acquire imaging data from different areas of the imaging subject 16. In some embodiments, the surface coils can be transmit coils or can be transmit/receive coils. Various combinations of transmit coils, receive coils, and/or transmit/receive coils can be embedded into the coils apparel.

In some embodiments, the coils of the two or more items of clothing are coupled to define a combined array of coils covering a larger area of the body. For example, the two items of clothing can include the coil shirt 40 and the coil trousers 41. The coils in the shirt 40 are coupled with the coils in the trousers 41 to define a combined array of coils spanning substantially the entire human body except for the head, feet, and hands. Additional items of clothing such as the cap 42, socks, and gloves, mittens, or the like can also be coupled into the combined array.

Instead of a plurality of coils disposed on or in an item of clothing, a radio frequency surface coil 44 or coil array not embedded in clothing can also be employed. Regardless of the particular magnetic resonance signal receive apparatus used, the main magnetic field coils 20 produce a main B₀ magnetic field. A magnetic resonance imaging controller 50 operates magnetic field gradient controllers 52 to selectively energize the magnetic field gradient coils 30, and operates a radio frequency transmitter 54 coupled to the radio frequency coil 32 as shown, or coupled to one or more of the coils apparel 40, 41, 42 or the surface coil 44, to selectively inject radio frequency excitation pulses into the subject 16. By selectively operating the magnetic field gradient coils 30 and the radio frequency coil 32 magnetic resonance is generated and spatially encoded in at least a portion of a region of interest of the imaging subject 16. By applying selected magnetic field gradients via the gradient coils 30, a selected k-space trajectory is traversed, such as a Cartesian trajectory, a plurality of radial trajectories, or a spiral trajectory.

During imaging data acquisition, the magnetic resonance imaging controller 50 operates a radio frequency receiver 56 coupled to one or more of the items of coil apparel 40, 41, 42, or to the radio frequency coils 44, to acquire magnetic resonance samples that are stored in a magnetic resonance data memory 60. The imaging data are reconstructed by a reconstruction processor 62 into an image representation. In the case of k-space sampling data, a Fourier transform-based reconstruction algorithm can be employed. Other reconstruction algorithms, such as a filtered backprojection-based reconstruction, can also be used depending upon the format of the acquired magnetic resonance imaging data. For SENSE imaging data, the reconstruction processor 62 reconstructs folded images from the imaging data acquired by each of the radio frequency coils, and then combines the folded images along with coil sensitivity parameters to produce an unfolded reconstructed image.

The reconstructed image generated by the reconstruction processor 62 is stored in an images memory 64, and can be displayed on a user interface 66, stored in non-volatile memory, transmitted over a local intranet or the Internet, viewed, stored, manipulated, or so forth. The user interface 66 also includes one or more operator controls such as a keyboard 68, a scanner control panel, or the like by which a radiologist, technician, or other operator of the magnetic resonance imaging scanner 10 communicates with the magnetic resonance imaging controller 50 to select, modify, and execute magnetic resonance imaging sequences.

The described magnetic resonance imaging system is an example only. The radio frequency coils and coil arrays described herein can be used with substantially any type of magnetic resonance imaging scanner, including but not limited to horizontal bore scanners, vertical bore scanners, open scanners, and so forth.

With reference to FIG. 2A, one embodiment of the surface coil 44 includes a thin, flexible printed circuit board 80 on which is disposed printed circuitry 82 defining a radio frequency antenna. The printed circuit board 80 is sandwiched between two foam layers 84, 86 that provide protection for the printed circuit board 80 and comfort for the imaging subject 16. Outer cover layers 88, 90 disposed outside the foam layers 84, 86 insulate the surface coil 44 against water, moisture, body fluids, and other forms of contamination. The outer cover layers 88, 90 are preferably made of a medical grade urethane, an expanded polytetraflouroethylene (i.e., expanded PTFE, available from W. L. Gore & Associates, Inc., Newark, Del.), a polyvinyl chloride (PVC) material, or the like.

Cleaning and disinfecting the surface coil 44 between uses, for example using a 10% Clorox solution, helps prevent the spread of pathogens from patient to patient. Depending upon where the surface coils are used, they may come into contact with or even become immersed in blood, urine, vomit, or other body fluids. Hence, to further reduce the likelihood of spreading infectious microbes, an anti-microbial agent 92 is preferably incorporated into the outer cover layers 88, 90. In FIG. 2A, the anti-microbial agent is diagrammatically represented by discrete dots; however, the anti-microbial agent is preferably a substance that is incorporated into the plastic resin used in forming the outer cover layers 88, 90 and is incorporated substantially uniformly throughout the cover layers 88, 90. In some embodiments, the anti-microbial agent is an anti-microbial resin additive (anti-microbial resin additives, fiber additives, paints and coatings are available, for example, from Microban International, Ltd., 275 Madison Avenue, Suite 3700, New York, N.Y. 10016). In some contemplated embodiments, the outer cover layers are an outer crust formed on the foam layers during formation of the foam layers. In these embodiments, the anti-microbial agent is preferably added into the resin used to form the foam layers 84, 86. In yet other contemplated embodiments, the outer cover layers 88, 90 are fabric layers formed of fibers incorporating an anti-microbial additive. The anti-microbial properties of the treated fabric enhance the acceptability of fabric covers in applications in which the coils are likely to be soiled by body fluids from the patient.

With reference to FIG. 2B, an alternative surface coil 44′ includes a printed circuit board 80′, antenna-defining printed circuitry 82′, foam layers 84′, 86′, and outer cover layers 88′, 90′ corresponding to the same-named components of the surface coil 44. In the surface coil 44′, however, the outer cover layers 88′, 90′ do not have an anti-microbial agent incorporated therein. Rather, anti-microbial coatings 92′, 94′ are applied to the outer surfaces of the outer cover layers 88′, 90′, respectively. The fabric layers can also be protected with stain repellants and other surface treatments.

The surface coils 44, 44′ incorporating an anti-microbial agent 92, 92′, 94′ advantageously reduce the likelihood of spreading infectious pathogens between patients. However, the coils 44, 44′ are inconvenient for the patient, appear unfamiliar to the patient, and their placement in contact with the patient or in close proximity thereto may be alarming to the patient. These issues become more acute as the number of surface coils increases, for example in the case of an array of surface coils disposed all the way around the torso of the patient. The use of such coil arrays is becoming more prevalent as imaging techniques such as phased-array imaging, SENSE imaging, and other multiple receive coil imaging techniques gain popularity. Hence, one or more of the items of coil apparel 40, 41, 42 is suitably employed for imaging employing large coil arrays for imaging large areas of the imaging subject 16.

With reference to FIG. 3A, the coil shirt 40 includes an item of clothing, namely a shirt 102 in the coil apparel 40, that is made of one or more layers of stretchable fabric. A plurality of radio frequency coils 104 are attached in or on the stretchable shirt 102 and define an array of radio frequency antennas. Other clothing items are also contemplated such as pants or trousers 41, a vest, one or a pair of socks, gloves, or mittens, the cap 42, a jump-suit, and the like as may be appropriate to cover the region or regions of the patient to be imaged.

As shown in FIG. 3B, an advantage of the stretchable radio frequency coil apparel 40 is that it readily adapts to differently sized and shaped imaging subjects. FIG. 3B diagrammatically shows the radio frequency coil apparel 40 worn by a larger, more robust imaging subject 16′ as compared with the imaging subject 16 of FIG. 3A. In the coil apparel 40, the radio frequency coils 104 do not themselves stretch to accommodate the more robust imaging subject 16′; rather, the fabric of the shirt 102 between the coils 104 stretches to accommodate the more robust imaging subject 16′. As a result, the radio frequency coils 104 substantially maintain their shape and size, but are more spread apart relative to one another when the shirt 102 is worn by the large and robust imaging subject 16′ as compared with when the shirt 102 is worn by the smaller and thinner imaging subject 16. While the radio frequency coils 104 preferably do not stretch in the plane of the clothing layers, they are preferably flexible transverse to the plane to provide bending to accommodate differently sized and shaped imaging subjects. Because the radio frequency coils 104 do not stretch, tuning parameters of the radio frequency coils 104 generally do not change significantly when the shirt 102 is worn by differently sized and shaped imaging subjects.

With reference to FIGS. 1, 3A, and 3B, optionally the radio frequency coil shirt apparel 40 further includes an electronic identification tag 106. The electronic identification tag outputs a unique wireless identification signal 108 that is used by the magnetic resonance imaging controller 50 or by the associated radiologist, technician, or other scanner operator to identify and verify that the imaging subject 16 is the intended imaging subject for the magnetic resonance imaging procedure about to be performed. The identifier tag 106 is used to associate the patient and coil identification with the resultant images. Although not shown in the drawings, it is further contemplated that the radio frequency coil apparel may also include air- or water-filled cooling tubes, Peltier devices, holes, slits, or other features that promote cooling of the radio frequency coils 104 and the imaging subject 16.

With reference to FIG. 4, a portion of an example multiple layer fabric of an item of clothing incorporating a coil array is shown. In this example, the item of clothing includes four layers of fabric: two coil attachment layers 110, 112 on which radio frequency coils 114 are attached, and two outer insulating layers 120, 122 that insulate the radio frequency coils 114 against water, moisture, body fluids, and other forms of contamination. The two inner coil attachment layers 110, 112 are typically made of natural fiber such as cotton or another comfortable fabric. The two outer insulating layers 120, 122 can be made of a dense nylon, expanded PTFE, or other water-resistant fabric or, for stronger protection against fluid penetration, can be made of a plastic or rubber “raincoat”-type material. In some embodiments, the two outer insulating layers 120, 122 include an anti-microbial agent incorporated into the material of the outer insulating layers 120, 122 or coated onto the outer insulating layers 120, 122.

Each radio frequency coil 114 includes a printed circuit board 130 on which printed circuitry 132 defining a radio frequency antenna is disposed. The printed circuit board 130 is preferably not stretchable in the plane of the supporting fabric 110, 112. However, the printed circuit board 130 is preferably bendable to accommodate curvature of the fabric in conforming with the imaging subject.

In the embodiment illustrated in FIG. 4, the printed circuit board 130 also supports an electronics module 134 coupled with the printed circuit antenna 132. The electronics module 134 may contain, for example: a pre-amplifier with matching circuitry to provide a high output impedance as seen by the coil; radio frequency baluns, traps, or the like for suppressing induced currents; detuning circuitry for detuning the coil from the magnetic resonance frequency during the transmit phase of magnetic resonance imaging; safety interlock circuitry; remotely controllable tuning circuitry; and the like. The electronics module 134 also outputs an output signal corresponding to the received magnetic resonance signal. In the embodiment illustrated in FIG. 4, the electronics module 134 outputs a wireless electromagnetic signal. Alternatively, the electronics module 134 can contain an electro-optic device that outputs a light signal to optical fibers embedded in the attachment layers 110, 112 or in other layers of the fabric. The embedded optical fibers carrying optical signals from the coils 114 can, for example, be collected into a pigtail that couples with a fiber coupler optically connected with the radio frequency receiver 56. In yet other embodiments, the electronics module 134 transmits an electrical output signal to conductive wires embedded in the attachment layers 110, 112 or in other layers of the fabric. The embedded conductive wires are collected at an electrical connector that connects with a cable leading to the radio frequency receiver 56. Alternatively, the optical or electrical signals are multiplexed in the time and/or frequency domain.

In some embodiments, the coils 114 have transmit capability. In these embodiments, the electronics module 134 typically includes a transmit/receive drive such as PIN diode switch/preamplifier circuitry. Alternatively, transmit capability can be added to the coils apparel by making one or more of the coils 114 dedicated transmit coils having transmit capability for producing magnetic resonance excitation.

The radio frequency coils 114 attached to the attachment layer 110 are staggered in the plane of the layer 110 respective to the radio frequency coils 114 attached to the attachment layer 112. If the fabric is lightly stretched, for example because the imaging subject is small and thin, then the radio frequency coils of only one of the attachment layers 110, 112 may provide sufficient coverage for the multi-coil imaging. In such a situation, the radio frequency coils of only one of the two attachment layers 110, 112 may be operated. On the other hand, if the fabric is substantially stretched, for example because the imaging subject is large and robust, then the radio frequency coils 114 of both attachment layers 110, 112 may be used to provide sufficient coverage for the multi-coil imaging. In that situation, the radio frequency coils of both attachment layers 110, 112 are suitably operated to provide sufficient coverage for the multi-coil imaging.

With reference to FIG. 5, it is to be appreciated that the item of clothing can be something other than the shirt 102 of FIGS. 3A and 3B. The item of clothing with which the coils array is attached can in general be a shirt, a vest, pants or trousers, a sock, a glove, a mitten, a cap, or substantially any other type of clothing. In FIG. 5, radio frequency coil apparel 200 includes a sock 202 in which are embedded three coil arrays 204, 206, 208 each having coils of a different coil size or coil characteristic. The coils of the coil array 204 are disposed outermost, cover the foot and ankle regions, and have the largest coil size. The coils of the coil array 206 are smaller and also cover the foot and ankle regions. The coils of the coil array 208 are smallest and are disposed innermost, and moreover cover only the foot region but not the ankle region. Each of the three coil arrays 204, 206, 208 are preferably disposed on a separate layer of fabric of the sock 202, although in some embodiments they may be interspersed amongst one another in a single layer. The magnetic resonance controller 50 suitably operates a selected one of the three coil arrays 204, 206, 208 that is optimal for the type of imaging being performed. In some embodiments an anti-microbial agent is incorporated into the fabric of the sock 202 to provide anti-microbial protection.

In the preceding embodiments, the radio frequency coils have been printed circuits disposed on substantially non-stretchable, albeit optionally flexible, printed circuit boards. Flexibility to allow the stretchable item of clothing to comport with differently sized and shaped imaging subjects is provided at gaps between the individual radio frequency coils, by relative movement of different fabric layers, and optionally by bending of individual coils.

With reference to FIG. 6, a portion of multiple layer fabric of an item of clothing incorporating a coil array is shown. In this example, the item of clothing includes fabric layers 300 into which flexible conductive wires 302 are embedded or intertwined. The flexible conductive wires 302 define the radio frequency antennae of the coils array. Preferably, tuning circuitry (not shown) such as varactor diodes, preamplifiers, and the like, are embedded into an area of the item of clothing which is unlikely to be stretched significantly by the imaging subject. The tuning circuitry is coupled with the flexible conductive wires 302 to correct the tuning of the coils array for resonance frequency changes introduced by extension or other deformation of the flexible conductive wires 302 due to stretching of the fabric layers 300 to fit differently sized and shaped imaging subjects. In other embodiments, the tuning circuitry is remotely located and coupled with the flexible conductive wires 302 by a cable connected to the item of clothing. In some embodiments, the fibers of the fabric layers incorporate an anti-microbial agent. If the flexible conductive wires 302 include an insulating coating or sheath, the insulating coating or sheath also preferably incorporates an anti-microbial agent.

With reference to FIGS. 7A and 7B, radio frequency coil apparel 400 includes a vest, shirt, or other item of clothing covering the torso 16t of the imaging subject 16. The item of clothing includes an inflatable layer or bladder 402 having a plurality of radio frequency coils 404 disposed in or on the inflatable layer 402. In the illustrated embodiment of in FIGS. 7A and 7B, the coils 404 are disposed on an outer surface of the inflatable layer 402. FIG. 7A shows the situation before inflation of the inflatable layer 402. Before inflation, the coils 404 are not arranged in any particular geometry respective to the imaging subject torso 16t. FIG. 7B shows the situation after inflation of the inflatable layer 402. The inflation causes the inner surface of the inflatable layer 402 to press up against and conform with the contours of the imaging subject torso 16t. The inflation also causes the outer surface of the inflatable layer 402 to become substantially rigid such that the radio frequency coils 404 substantially conform to a pre-selected geometry relative to the imaging subject torso 16t. The amount of inflation should be enough to impose a reasonably fixed geometry on the coils 404, but not enough to produce an uncomfortable amount of pressure on the imaging subject 16t. A pressure gauge is optionally used to ensure that the inflatable layer 402 is inflated to a specific pressure value providing the desired pre-selected geometry.

With reference to FIGS. 7C and 7D, an advantage of the radio frequency coil apparel 400 is that the relative pre-selected geometry of the coils 404 relative to the imaging subject is substantially independent of the size and shape of the imaging subject. Thus, in FIGS. 7C and 7D the same inflatable radio frequency coil apparel 400 of FIGS. 7A and 7B is placed on a thinner, smaller imaging subject torso 16t′. FIG. 7C shows the uninflated configuration, in which the coils 404 are not arranged in any particular geometry respective to the imaging subject torso 16t′. FIG. 7D shows the inflated configuration: the inner surface presses up against and conforms with the thinner, less rotund torso 16t′, while the outer surface inflates to place the radio frequency coils 404 into substantially the same pre-selected geometry as was obtained in FIG. 7B for the rotund torso 16t.

In any of the described embodiments or their equivalents, an insulating layer or layers can be provided to insulate the radio frequency coils. However, it may be preferred to omit such a moisture barrier layer and instead rely upon shutoff of the coils in the event of fluid contamination. In such cases, all layers of the item of clothing supporting the radio frequency coils can be natural fabric or another comfortable fabric material. In any of the described embodiments or their equivalents, an anti-microbial agent can be incorporated into portions of the radio frequency receiving apparatus that contact the imaging subject 16. Rigid coils or coil assemblies can include rigid plastic encasements which include an anti-microbial resin additive. Moreover, an anti-microbial agent is optionally incorporated into other portions of the magnetic resonance imaging system that are contacted by the imaging subject 16 or by a radiologist, technician, or other operator. For example, the keyboard 68 or other operator control, the gantry or housing 12, the patient support 18, or the like can incorporate an anti-microbial agent. Similarly, pads used to position or comfort the imaging subject 16 can incorporate an anti-microbial agent. Incorporating an anti-microbial agent into surfaces contacted by the imaging subject 16 or the radiologist helps prevent the spread of infectious pathogens between patients or between a patient and the radiologist.

The invention has been described with reference to the preferred embodiments. Obviously, modifications and alterations will occur to others upon reading and understanding the preceding detailed description. It is intended that the invention be construed as including all such modifications and alterations insofar as they come within the scope of the appended claims or the equivalents thereof.

1. A radio frequency apparatus for at least one of (i) receiving and (ii) exciting a magnetic resonance signal, the apparatus comprising:
an item of clothing comprising one or more layers that are stretchable to comport with differently sized and shaped imaging subjects; and
a plurality of radio frequency coils attached to one or more layers of the item of clothing, the coils being relatively movable with respect to one another responsive to stretching of the stretchable layers.

2. The radio frequency apparatus as set forth in claim 1, wherein the radio frequency coils are generally flexible planar coils in which the plane of the coil lies generally parallel to the layer or layers to which the coil is attached.

3. The radio frequency apparatus as set forth in claim 1, wherein each radio frequency coil includes:
a printed circuit board lying generally parallel to the layer or layers to which the coil is attached, the printed circuit board including a printed circuit defining a radio frequency antenna.

4. The radio frequency apparatus as set forth in claim 1, wherein the plurality of radio frequency coils include:
a plurality of first radio frequency coils attached to the item of clothing, the first radio frequency coils having a first coil size or characteristic; and
a plurality of second radio frequency coils attached to the item of clothing, the second radio frequency coils having a second coil size or characteristic.

5. The radio frequency apparatus as set forth in claim 4, wherein the first coil size or characteristic is different from the second coil size or characteristic.

6. The radio frequency apparatus as set forth in claim 4, wherein (i) the plurality of first radio frequency coils are attached to a first stretchable layer, and (ii) the plurality of second radio frequency coils are attached to a second stretchable layer different from the first stretchable layer.

7. The radio frequency apparatus as set forth in claim 4, wherein the apparatus further includes:
a means for selectively employing one of (i) the plurality of first radio frequency coils and (ii) the plurality of second radio frequency coils for receiving the magnetic resonance signal.

8. The radio frequency apparatus as set forth in claim 1, wherein the plurality of radio frequency coils include:
a plurality of flexible conductive wires woven into the one or more stretchable layers of the item of clothing, the flexible conductive wires defining a plurality of radio frequency antennas.

9. The radio frequency apparatus as set forth in claim 1, wherein the item of clothing is selected from a group consisting of a shirt, a vest, pants or trousers, a sock, a glove, a mitten, a jump-suit, and a cap.

10. The radio frequency apparatus as set forth in claim 1, further including:
an anti-microbial agent disposed on or in one or more of the layers of the item of clothing.

11. The radio frequency apparatus as set forth in claim 1, wherein the item of clothing further includes:
at least one layer formed of fibers incorporating or coated with an anti-microbial agent.

12. The radio frequency apparatus as set forth in claim 1, wherein the item of clothing further includes:
at least one layer formed of an expanded PTFE material incorporating or coated with an anti-microbial agent.

13. The radio frequency apparatus as set forth in claim 1, wherein the layers of the item of clothing include:
at least one water-resistant layer insulating the plurality of radio frequency coils.

14. The radio frequency apparatus as set forth in claim 1, further including:
two or more items of clothing each including a plurality of radio frequency coils, the coils of the two or more items of clothing being coupled together to define a combined coil array.

15. The radio frequency apparatus as set forth in claim 1, further including:
an electronic identification tag attached to the item of clothing.

16. The radio frequency apparatus as set forth in claim 1, wherein the layers of the item of clothing include:
an inflatable layer, the plurality of radio frequency coils being disposed in or on the inflatable layer and substantially conforming to a pre-selected geometry responsive to inflation of the inflatable layer.

17. The radio frequency apparatus as set forth in claim 1, wherein the plurality of radio frequency coils attached to one or more layers of the item of clothing include at least one radio frequency transmit coil for exciting magnetic resonance and a plurality of radio frequency receive coils for receiving the excited magnetic resonance.

18. A radio frequency apparatus for at least one of (i) receiving and (ii) exciting a magnetic resonance signal, the apparatus comprising:
at least one radio frequency antenna; and
a structure disposed on or around the at least one radio frequency antenna, the structure including an anti-microbial agent disposed on or incorporated into the structure.

19. The radio frequency apparatus as set forth in claim 18, wherein the structure comprises:
one or more clothing layers defining an item of clothing, the clothing layers being stretchable to comport with differently sized and shaped imaging subjects.

20. The radio frequency apparatus as set forth in claim 19, wherein the each radio frequency antenna comprises:
one or more printed circuit boards, the at least one radio frequency antenna being defined by printed circuitry of the one or more printed circuit boards, the one or more printed circuit boards being attached to at least one clothing layer.

21. The radio frequency apparatus as set forth in claim 19, wherein the one or more clothing layers comprise:
a water resistant layer insulating the at least one radio frequency antenna, the anti-microbial agent being disposed on or incorporated into the water resistant layer.

22. The radio frequency apparatus as set forth in claim 19, wherein the one or more layers include:
an inflatable layer surrounding an imaging subject imaged by the at least one radio frequency antenna, the at least one radio frequency antenna being disposed in or on the inflatable layer and assuming a selected position relative to the imaging subject responsive to inflation of the inflatable layer.

23. A magnetic resonance imaging scanner for imaging an imaging subject, the scanner comprising:
a main magnet generating a substantially spatially and temporally constant magnetic field in an examination region, the main magnet being housed in a gantry;
magnetic field gradient coils housed in the gantry and generating selected magnetic field gradients in the examination region;
a subject support for supporting the subject in the examination region;
at least one radio frequency coil arranged proximate to the imaging subject in the examination region;
an operator control contacted by an associated scanner operator; and
an anti-microbial agent disposed on or incorporated into at least one of (i) the at least one radio frequency coil, (ii) the gantry, (iii) the subject support) and (iv) the operator control.
